(19) United States
(12) Patent Application Publication
(10) Pub. No.: US 2019/0151293 A1
(43) Pub. Date: May 23, 2019

(54) PHARMACEUTICAL COMPOSITION COMPRISING A NON-PURINE SELECTIVE INHIBITOR OF XANTHINE OXIDASE AND METHOD FOR THE PREPARATION THEREOF

(71) Applicant: PHARMATHEN S.A., PALLINI-ATTIKIS (GR)

(72) Inventors: EVANGELOS KARAVAS, PALLINI ATTIKIS (GR); EFTHYMIOS KOUTRIS, PALLINI ATTIKIS (GR); VASILIKI SAMARA, PALLINI ATTIKIS (GR); IOANNA KOUTRI, PALLINI ATTIKIS (GR); ANASTASIA KALASKANI, PALLINI ATTIKIS (GR); CHRISTINA KIZIRIDI, PALLINI ATTIKIS (GR); MORFIS ABATZIS, PALLINI ATTIKIS (GR); KATERINA TSILIOUKA, PALLINI ATTIKIS (GR)

(73) Assignee: PHARMATHEN S.A., PALLINI-ATTIKIS (GR)

(21) Appl. No.: 16/308,908

(22) PCT Filed: Jun. 26, 2017
(86) PCT No.: PCT/EP2017/025179

§ 371 Date: Dec. 11, 2018

(30) Foreign Application Priority Data
Jun. 30, 2016 (GR) 20160100353

(51) Int. Cl. A61K 31/426; A61K 9/20

(52) U.S. Cl. CPC A61K 31/426; A61K 9/2009; A61K 9/2095

(57) ABSTRACT

The present invention relates to a stable pharmaceutical formulation of solid dosage forms for oral administration comprising a therapeutically effective amount of a non-purine selective inhibitor of xanthine oxidase, in particular Febuxostat and an effective amount of an alkalizing agent. It also relates to a process for the preparation thereof.

### TECHNICAL FIELD OF INVENTION

The present invention relates to a stable pharmaceutical formulation for oral administration containing a therapeutically effective quantity of a non-purine selective inhibitor of xanthine oxidase such as Febuxostat and a method for the preparation thereof.

### BACKGROUND OF THE INVENTION

Uric acid is formed from the breakdown of certain chemicals (purines) in the body. Hyperuricemia occurs when the body produces more uric acid than it can eliminate. The uric acid forms crystals in joints (gouty arthritis) and tissues, causing inflammation and pain. Elevated blood uric acid levels also can cause kidney disease and kidney stones.

Uric acid is the end product of purine metabolism in humans and is generated in the cascade of hypoxanthine to xanthine to uric acid. Both steps in the above transformations are catalyzed by xanthine oxidase (XO). Febuxostat is a 2-arylthiazole derivative that achieves its therapeutic effect of decreasing serum uric acid by selectively inhibiting XO. Febuxostat has been shown to inhibit both the oxidised and reduced forms of XO. At therapeutic concentrations febuxostat does not inhibit other enzymes involved in purine or pyrimidine metabolism.

For many years allopurinol has been the most widely used urate-lowering agent. Febuxostat which is structurally different from allopurinol by lacking the purine ring is a more selective and potent inhibitor of XO and has no effect on other enzymes involved in purine or pyrimidine metabolism.

The chemical name of Febuxostat is 2-(3-cyano-4-isobutoxyphenyl)-4-methyl-1,3-thiazole-5-carboxylic acid. The molecular formula is C₁₆H₁₆N₂O₃S corresponding to a molecular weight of 316.374. It is a white crystalline powder. Febuxostat is practically insoluble in water, sparingly soluble in ethanol, soluble in dimethylsulfoxide and freely soluble in dimethylformamide.

Febuxostat exhibits polymorphism. The crystal forms of Febuxostat disclosed in EP1020454, namely anhydrate A, anhydrate B, anhydrate C, hydrate G, a solvate with methanol (Form D) and anhydrated form K are the most known crystalline forms

WO-A-2012/153313 discloses an immediate-release Febuxostat composition comprising an inert carrier covered with at least one layer containing Febuxostat in a micronized form, a hydrophilic polymer and, optionally, a surfactant.

WO-A-2014/125504 discloses an immediate release tablet comprising Febuxostat and an acid component in an amount of from 0.05% to 2% by weight of the tablet.

Although each of the patents above represents an attempt to provide stable Febuxostat compositions for oral administration, there still remains the need in the art for alternative formulations with enhanced dissolution and adequate chemical and physical characteristics.

### SUMMARY OF THE INVENTION

It is, therefore, an object of the present invention to provide a thermodynamically stable and efficient product comprising a non-purine selective inhibitor of xanthine oxidase such as Febuxostat suitable for oral administration.

The present invention aims at developing a formulation that not only matches the physical and chemical attributes of the reference product but also overcomes the disadvantages associated with the prior art compositions.

Further object of the present invention is to provide a film-coated tablet comprising Febuxostat as an active ingredient, which is bioavailable and with sufficient self-life.

A major object of the present invention is the selection of the optimal combination of pharmaceutical acceptable excipients, the effective drug substance particle size distribution and the method of preparation of final product in order to achieve the appropriate dissolution profile and stability for the finished dosage form. Said dosage form affords predictable and reproducible drug release rates in order to achieve better treatment to a patient.

A further approach of the present invention is to provide a tablet composition for oral administration comprising Febuxostat which is manufactured through a fast, simple and cost-effective process.

According to another embodiment of the present invention, a process for the preparation of a solid dosage form for oral administration, containing a non-purine selective inhibitor of xanthine oxidase and in particular Febuxostat as an active ingredient and an effective amount of an alkalizing agent is provided, which comprises the following steps:
Sifting and mixing the internal phase excipients;
Kneading with water;
Drying;
Sizing the granules;
Mixing with external phase excipients;
Adding at least one lubricant and mixing;
Compressing the resulted mixture into a tablet dosage form;
Optionally applying a film-coating on the core.

Other objects and advantages of the present invention will become apparent to those skilled in the art in view of the following detailed description.

### DETAILED DESCRIPTION OF THE INVENTION

For the purposes of the present invention, a pharmaceutical composition comprising an active ingredient is considered to be “stable” if said ingredient degrades less or more slowly than it does on its own and/or in known pharmaceutical compositions.

As already mentioned the main object of the present invention is to provide a stable pharmaceutical composition of Febuxostat for oral administration that is simple to manufacture, bioavailable, cost effective and possesses good pharmacotechnical properties.

Febuxostat exhibits polymorphism. Polymorphism is a phenomenon relating to the occurrence of different crystal forms for one molecule. There may be several different crystalline forms for the same molecule with distinct crystal structures and varying in physical properties like melting point, XRPD spectrum and IR-spectrum. These polymorphs are thus distinct solid forms which share the molecular formula of the compound from which the crystals are made up; however, they may have distinct advantageous physical properties which can have a direct effect on the ability to process and/or manufacture the drug substance as well as on drug product stability, dissolution, and bioavailability. These distinct physical properties of different polymorphs of the same compound can render different polymorphs more or less useful for a particular purpose, such as for pharmaceutical formulation.

Form C was selected in the present invention as this form exhibits good solubility and fewer problems with regard to polymorphic conversion during preparation and/or typical formulation conditions and storage.

Solubility, the phenomenon of dissolution of solute in solvent to give a homogenous system, is one of the important parameters to achieve desired concentration of drug in systemic circulation for desired (anticipated) pharmacological response. Febuxostat belongs to class II according to the Biopharmaceutical Classification System. It is permeable but relatively insoluble, and is considered not such good clinical candidate without the use of enhanced formulation techniques aimed at increasing solubility or rate of dissolution. Solubility enhancement is a major challenge for formulation development. Any drug to be absorbed must be present in the form of solution at the site of absorption. Various techniques are used for the enhancement of the solubility of poorly soluble drugs which include physical and chemical modifications of drug and other methods like particle size reduction, crystal engineering, salt formation, solid dispersion, use of surfactant, complexation, and addition of alkalizing or acidifying agents. Selection of solubility improving method depends on drug property, site of absorption, and required dosage form characteristics.

Febuxostat solubility is pH dependent; in alkalic media the solubility is higher. Therefore, an alkalizing agent may enhance dissolution rate. The alkalizer is used to create a microenvironment in the formulation to optimize drug release after the formulation is in a hydrated media. The alkalizers used in the present invention are capable of raising the pH of the micro-environment of the hydrated formulation to a pH greater of the starting pH of the media.

Alkalizing agents used in the present invention include, for example, magnesium oxide, dibasic calcium phosphate, tricalcium phosphate, calcium carbonate and are used in an amount 3-10% (w/w). Preferably, magnesium oxide is used in the present invention.

The particle size of the API is a critical parameter that can affect the solubility of low solubility API's such as Febuxostat. The specific surface area is increased with decreasing particle size of the drug, resulting in an increase in dissolution rate. In most circumstances the dissolution rate of poorly soluble drugs is strongly related to the particle size distribution and thus the dissolution profile of the final product.

In order for a drug to have its effect after oral administration it must go into solution and then diffuse through the gut wall into the body. The first step in that process is the disintegration of the dosage form followed by dissolution of the active ingredient. One way to increase dissolution rate of poorly soluble drugs such as Febuxostat is to increase the surface available for dissolution by reducing particle size. It has been surprisingly found that the objects of the present invention are achieved when the formulation is prepared using Febuxostat with specific particle size, in particular wherein D90<30 μm.

The pharmaceutical compositions of the present invention may also contain one or more additional formulation excipients such as diluents, disintegrants, binders, lubricants, provided that they are compatible with the active ingredient of the composition, so that they do not interfere with it in the composition and in order to increase the stability of the drug and the self-life of the pharmaceutical product.

Diluents increase the bulk of a solid pharmaceutical composition, and may make a pharmaceutical dosage form easier for the patient and care giver to handle. Diluents for solid compositions include, for example, microcrystalline cellulose (MCC), dextrose, fructose, mannitol, maltodextrin, maltitol, lactose.

The dissolution rate of a compacted solid pharmaceutical composition in the patient's stomach may be increased by the addition of a disintegrant to the composition. Disintegrants include sodium starch glycolate, alginic acid, carboxymethylcellulose sodium, croscarmelose sodium, colloidal silicon dioxide (aerosil).

Solid pharmaceutical compositions that are compacted into a dosage form, such as a tablet, may include excipients whose function include helping to bind the active ingredient and other excipients together after compression. Binders for solid pharmaceutical compositions include hydroxyethyl cellulose, methylcellulose, hydroxypropyl cellulose (HPC), polydextrose, polyethylene oxide, povidone.

When a dosage form such as a tablet is made by the compaction of a powdered composition, the composition is subjected to pressure from a punch and dye. Some excipients and active ingredients have a tendency to adhere to the surfaces of the punch and dye, which can cause surface irregularities to the product. A lubricant can be added to the composition to reduce adhesion and ease the release of the product from the dye. Lubricants include talc, magnesium stearate, calcium stearate, glyceryl behenate.

The following examples illustrate preferred embodiments in accordance with the present invention without limiting the scope or spirit of the invention.

### EXAMPLES

The formulation development started with three composition trials comprising as ingredients API, lactose monohydrate, MCC, croscarmellose sodium, aerosil, HPC-L and magnesium stearate. For the first trial dry mixing procedure was chosen, while for the second and the third one wet granulation was selected using as solvent ethanol and water respectively.

TABLE 1
Compositions 1-3

| | Composition 1-3 |
|---|---|
| | % |
| Internal Phase | |
| Febuxostat | 15.69 |
| Lactose monohydrate | 15.00 |
| MCC | 60.31 |
| Croscarmellose sodium | 5.00 |
| HPC-L | 2.00 |
| Aerosil | 1.00 |
| External Phase | |
| Mg Stearate | 1.00 |
| Total for uncoated tablet | 100.00 |

The manufacturing process of Composition 1 includes dry mixing of ingredients. The preparation steps followed are presented below:
Raw materials dispensing;
Sifting the raw materials;
Blending API with internal phase excipients;
Lubrication with Magnesium stearate;
Compression.

The manufacturing process of Composition 2 & 3 includes wet granulation using ethanol & water respectively as solvent. The preparation steps followed are presented below:
Raw materials dispensing;
Sifting the raw materials;
Blending API with internal phase excipients;
Kneading with the appropriate quantity of ethanol (Composition 2) or water (Composition 3);
Drying;
Sizing the granules;
Lubrication with Magnesium stearate;
Compression.

The physicochemical characteristics achieved are presented in table 2 below:

TABLE 2
Results of Compositions 1-3

| | | Composition | Composition | Composition |
|---|---|---|---|---|
| Test parameters | Target | 1 | 2 | 3 |
| Hardness | 180-300 N | 173 N | 112 N | 155 N |
| Disintegration | 4′00″-5′00″ | 5′00″-5′18″ | 1′00″-1′44″ | 1′25″-1′44″ |
| time | | | | |

Furthermore, Compositions 1, 2 and 3 were tested for their dissolution rate in dissolution media of 0.05 M Phosphate Buffer, pH 6.0 at 75 rpm, II (Paddle).

TABLE 3
Dissolution profile of Compositions 1-3

| | Compositions | | | | | |
|---|---|---|---|---|---|---|
| | 1 | | 2 | | 3 | |
| Time | % | % | % | % | % | % |
| interval | Dissolved | RSD | Dissolved | RSD | Dissolved | RSD |
| 5 min | 46.40 | 4.15 | 31.62 | 1.54 | 27.66 | 4.37 |
| 10 min | 46.49 | 0.93 | 45.54 | 0.12 | 40.30 | 3.12 |
| 15 min | 48.37 | 4.35 | 48.93 | 0.71 | 47.12 | 1.83 |
| 20 min | 49.56 | 0.41 | 55.09 | 1.16 | 54.13 | 0.89 |
| 30 min | 52.54 | 0.18 | 61.09 | 1.24 | 60.46 | 0.95 |
| 45 min | 60.31 | 0.25 | 66.46 | 1.52 | 66.02 | 0.67 |

As Febuxostat solubility is pH dependent the solubility is higher in alkalic media. Therefore, it was decided to add an alkalizing agent into the internal phase excipients in order to enhance dissolution rate.

TABLE 4
Compositions 4-7

| | Composition | | | |
|---|---|---|---|---|
| | 4 | 5 | 6 | 7 |
| | % | | | |
| Internal Phase | | | | |
| Febuxostat | 15.69 | 15.69 | 15.69 | 15.69 |
| Lactose monohydrate | 17.00 | 17.00 | 17.00 | 17.00 |
| MCC | 53.31 | 53.31 | 53.31 | 53.31 |
| Croscarmellose sodium | 5.00 | 5.00 | 5.00 | 5.00 |
| HPC-L | 2.00 | 2.00 | 2.00 | 2.00 |
| Magnesium oxide (magnesia 211) | 5.00 | — | — | — |
| Dibasic calcium phosphate | — | 5.00 | — | — |
| Tricalcium phosphate (TRI-CAFOS) | — | — | 5.00 | — |
| Calcium carbonate | — | — | — | 5.00 |
| Aerosil | 1.00 | 1.00 | 1.00 | 1.00 |
| External Phase | | | | |
| Mg Stearate | 1.00 | 1.00 | 1.00 | 1.00 |

| | |
|---|---|
| Total for uncoated tablet | 100.00 |

The manufacturing process of Compositions 4-7 includes dry mixing of ingredients. The preparation steps followed are presented below:
Raw materials dispensing;
Sifting the raw materials;
Blending API with internal phase excipients;
Lubrication with Magnesium stearate;
Compression.

Compositions 4-7 were tested for their dissolution rate (buffer, pH=6, paddles, 75 rpm)

TABLE 5
Dissolution profile of Compositions 4-7

Compositions

| | | | |
|---|---|---|---|
| 4 | 5 | 6 | 7 |

| | |
|---|---|
| Time interval | % Dissolved |

| | | | | |
|---|---|---|---|---|
| 5 min | 28.58 | 21.97 | 47.49 | 39.04 |
| 10 min | 54.11 | 30.15 | 57.62 | 52.66 |
| 15 min | 66.32 | 40.2 | 62.87 | 60.42 |
| 20 min | 73.50 | 45.23 | 66.72 | 64.57 |
| 30 min | 80.87 | 51.23 | 71.74 | 68.69 |
| 45 min | 82.13 | 55.30 | 76.59 | 73.54 |

The physicochemical characteristics achieved are presented in table 6 below:

TABLE 6
Results of Compositions 4-7

| Test | | Compo- | Com- | Compo- | |
|---|---|---|---|---|---|
| parameters | Target | sition 4 | position 5 | sition 6 | Composition 7 |
| Hardness | 255 N | 206 N | 215 N | 230 N | 243 N |
| Disintegration | 3′-4′ | 0′54″ | 0′37″ | 0′41″ | 0′59″ |
| pH | 6.23 | 8.83 | 6.85 | 5.85 | 9.57 |

Composition 4 with magnesium oxide and Composition 7 with calcium carbonate as alkalizing agents gave the best dissolution profile.

Both Compositions 4 and 7 revealed pH above 8; therefore, it was decided to set a new target for pH since the high pH of the tablet facilitates the alkalic microenvironment that optimizes drug release after the formulation is in a hydrated media. In order to investigate the effective amount of the alkalizing agent further compositions were prepared, i.e 7a, 7b, 7c, 7d, 4a, 4b, 4c, 4d.

TABLE 7
Compositions 7, 7a, 7b, 7c, 7d, 4, 4a, 4b, 4c, 4d

| | % amount of | | | | |
|---|---|---|---|---|---|
| Compo- | alkalizing | | Dis- | | |
| sition | agent | alkalizing agent | integration | Hardness | pH |
| 7a | 1 | calcium carbonate | 0′22″ | 115 N | 6.45 |
| 7b | 3 | calcium carbonate | 0′36″ | 147 N | 7.12 |
| 7 | 5 | calcium carbonate | 0′59″ | 243 N | 9.57 |
| 7c | 8 | calcium carbonate | 00′63″ | 212 N | 10.64 |
| 7d | 10 | calcium carbonate | 01′74″ | 254 N | 12.4 |
| 4a | 1 | magnesium oxide | 0′43″ | 180 N | 5.58 |
| 4b | 3 | magnesium oxide | 0′49″ | 186 N | 6.97 |
| 4 | 5 | magnesium oxide | 0′54″ | 206 N | 8.83 |
| 4c | 8 | magnesium oxide | 01′24″ | 195 N | 9.87 |
| 4d | 10 | magnesium oxide | 02′29″ | 200 N | 11.45 |

The dissolution profile was examined for compositions with alkalic pH in the target range 8-11.

TABLE 8
Dissolution profile of Compositions 7, 7c, 4, 4c, 4d

| Time | Composition 7 | Composition 7c | Composition 4 | Composition 4c | Composition 4d |
|---|---|---|---|---|---|
| interval | % release | | | | |
| 5 min | 39.04 | 40.92 | 28.58 | 37.32 | 35.69 |
| 10 min | 52.66 | 62.85 | 54.11 | 50.74 | 48.63 |
| 15 min | 60.42 | 65.36 | 66.32 | 67.65 | 55.74 |
| 20 min | 64.57 | 67.64 | 73.50 | 70.74 | 64.38 |
| 30 min | 68.69 | 71.04 | 80.87 | 72.98 | 71.23 |
| 45 min | 73.54 | 72.43 | 82.13 | 79.43 | 73.69 |

The higher amount of alkalizing agent offers a faster dissolution rate at the early stages but does not improve solubility significant at the final dissolution points. Thus, the effective amount of alkalizing agent is set to be in the range of 5-10%.

The following DOE was focused on selecting among the two alkalizing agents and their optimum % amount and an appropriate disintegrant for improving the physicochemical characteristics. The amount of alkalizing agent remained 5% for all compositions.

TABLE 9
Compositions 7.1, 7.2, 7.3, 7.4, 7.5, 4.1, 4.2, 4.3, 4.4, 4.5

| | | Amount of | | | |
|---|---|---|---|---|---|
| Composition | Disintegrant | disintegrant | Alkalizing agent | Disintegration | Hardness |
| 7.1 | crospovidone | 5 | calcium carbonate | 04′15″ | 239 N |
| 4.1 | primojel | 5 | magnesium oxide | 05′19″ | 200 N |
| 4.2 | croscarmellose sodium | 8 | magnesium oxide | 01′18″ | 208 N |
| 4.3 | crospovidone | 5 | magnesium oxide | 04′26″ | 207 N |
| 7.2 | primojel | 5 | calcium carbonate | 06′42″ | 224 N |
| 7.5 | primojel | 8 | calcium carbonate | 03′43″ | 180 N |
| 4.4 | crospovidone | 8 | magnesium oxide | 02′21″ | 186 N |
| 4.5 | primojel | 8 | magnesium oxide | 01′19″ | 150 N |
| 7.3 | crospovidone | 8 | calcium carbonate | 01′24″ | 195 N |
| 7.4 | croscarmellose sodium | 8 | calcium carbonate | 02′29″ | 200 N |

The dissolution profile was investigated for the faster disintegrated compositions.

TABLE 10
Dissolution profile of Compositions 4.2, 4.3, 4.4, 4.5, 7.3, 7.4

| | Compo- | Compo- | Compo- | Compo- | Compo- | Compo- |
|---|---|---|---|---|---|---|
| | sition | sition | sition | sition | sition | sition |
| Time | 4.2 | 4.3 | 4.4 | 4.5 | 7.3 | 7.4 |
| interval | % Dissolved | | | | | |
| 5 min | 35.65 | 12.81 | 34.89 | 44.16 | 32.56 | 42.15 |
| 10 min | 57.11 | 28.47 | 52.3 | 55.64 | 45.87 | 55.12 |
| 15 min | 70.19 | 41.36 | 65.4 | 63.5 | 54.13 | 59.45 |
| 20 min | 75.56 | 49.67 | 75.4 | 70.45 | 60.18 | 60.18 |
| 30 min | 80.87 | 57.57 | 79.1 | 73.4 | 65.39 | 62.36 |
| 45 min | 83.15 | 64.13 | 81.2 | 77.65 | 74.12 | 70.47 |

The above compositions revealed that 8% of disintegrant improves the physicochemical characteristics of the tablets and improves the dissolution rate.

From the dissolution results of the DOE only Composition 4.2 containing croscarmellose sodium as disintegrant at 8% and magnesium oxide as alkalizing agent at 5% had significant higher dissolution rate than the other compositions.

Therefore, it was decided to investigate if Composition 4.2 which is a dry mixing can be further improved with respect to solubility; thus, a wet granulation process was followed.

Compositions 8 & 9 were prepared following a wet granulation process with magnesium oxide as alkalizing agent and croscarmellose sodium as a disintegrant at 8%, which was split between internal and external phase.

TABLE 11
Compositions 8 & 9

Composition

| | |
|---|---|
| 8 | 9 |

%

| | | |
|---|---|---|
| Internal Phase | | |
| Febuxostat | 15.69 | 15.69 |
| Lactose monohydrate | 17.00 | 15.00 |
| MCC | 53.31 | 52.31 |
| Croscarmellose sodium | 6.00 | 3.00 |
| HPC-L | 2.00 | 2.00 |
| Mg Oxide | 5.00 | 5.00 |
| Aerosil | 1.00 | 1.00 |
| External Phase | | |
| Croscarmellose sodium | 2.00 | 5.00 |
| Mg Stearate | 1.00 | 1.00 |

| | |
|---|---|
| Total for uncoated tablet | 100.00 |

The manufacturing process of Compositions 8 & 9 includes a wet granulation of internal phase ingredients. The preparation steps followed are presented below:
Raw materials dispensing;
Sifting the raw materials;
Blending API with internal phase excipients and water;
Drying the wet mass at 40° C.;
Addition of rest amount of croscarmellose sodium;
Lubrication with magnesium stearate.

The physicochemical characteristics achieved are presented in table 12 below:

TABLE 12
Results of Compositions 8 & 9

| Test parameters | Target | Composition 8 | Composition 9 |
|---|---|---|---|
| Hardness | 180-300 N | 172 N | 279 N |
| Disintegration time | 4′00″-5′00″ | 12′28″ | 6′00″ |

Disintegration time of Composition 8 was very high; therefore, the amount of disintegrant from the internal phase was split and moved to the external phase in Composition 9 so as to decrease disintegration time. The disintegration time was improved but remained still high; therefore, it was decided to transfer the amount of Aerosil to the external phase (Composition 10).

TABLE 13
Composition 10

| | Composition 10 |
|---|---|
| | % |
| Internal Phase | |
| Febuxostat | 15.69 |
| Lactose monohydrate | 15.00 |
| MCC | 52.31 |
| Croscarmellose sodium | 3.00 |
| HPC-L | 2.00 |
| Mg Oxide | 5.00 |
| External Phase | |
| Aerosil | 1.00 |
| Croscarmellose sodium | 5.00 |
| Mg Stearate | 1.00 |
| Total for uncoated tablet | 100 |

The manufacturing process of Composition 10 includes a wet granulation of internal phase ingredients. The preparation steps followed are presented below:
Raw materials dispensing;
Sifting the raw materials;
Blending API with internal phase excipients and water;
Drying the wet mass at 40° C.;
Addition of rest amount of croscarmellose sodium and aerosil;
Lubrication with magnesium stearate.

The physicochemical characteristics achieved are presented in table 14 below:

TABLE 14
Results of Composition 10
Composition 10

| | Results for | Results for 3% coated | |
|---|---|---|---|
| Test parameters | uncoated tablets | tablets | Target |
| Hardness | 236 N | 313 N | 180-350 N |
| Disintegration | 3′00″-3′23″ | 4′00′-4′50″ | 3′00″-5′00″ |
| time | | | |

The dissolution profile of Composition 10 was also investigated.

TABLE 15
Dissolution profile of Composition 10
Composition 10

| Time interval | % Dissolved |
|---|---|
| 5 min | 47.69 |
| 10 min | 68.36 |
| 15 min | 76.56 |
| 20 min | 80.98 |
| 30 min | 84.35 |
| 45 min | 90.40 |

Physical characteristics and dissolution profile of Composition 10 were satisfactory.

Compositions 1-10 were performed with Febuxostat polymorphic form C with an average particle size distribution of D(0,9) =60 μm. In order to investigate the effect of particle size to Febuxostat solubility further evaluation was designed based on Composition 10 with API of particle size of D (0, 9)=30 μm, (Composition 10.1) D (0, 9)=20 μm (Composition 10.2) and D (0, 9)=5 μm (Composition 10.3).

The dissolution results at buffer pH=6.0, paddles, 75 rpm are shown in table 16 below:

TABLE 16
Dissolution profile of Compositions 10, 10.1, 10.2, 10.3

| | Composition 10 | Composition 10.1 | Composition 10.2 | Composition 10.3 |
|---|---|---|---|---|
| Time | D (0, 9) = 60 μm | D (0, 9) = 30 μm | D (0, 9) = 20 μm | D (0, 9) = 5 μm |
| interval | % release | | | |
| 5 min | 47.69 | 50.92 | 55.92 | 57.32 |
| 10 min | 68.36 | 72.85 | 79.47 | 80.74 |
| 15 min | 76.56 | 85.36 | 86.83 | 87.65 |
| 20 min | 80.98 | 87.64 | 86.93 | 90.74 |
| 30 min | 84.35 | 91.04 | 91.11 | 92.98 |
| 45 min | 90.4 | 92.43 | 93.43 | 94.43 |

Comparing dissolution profiles of Compositions 10, 10.1, 10.2 and 10.3 it can be concluded that particle size of the active ingredient has an effect on dissolution properties and most particular on the early stages of the dissolution rate. At 30 minutes the PSD reduction enhances the dissolution rate above the target of 85% release for this time interval. Thus, the effective particle size is set to be in the range of D90: 5-30 μm.

Tablets of Composition 10 were placed in chambers under normal (25° C. /60% RH), intermediate (30° C./65% RH) and accelerated conditions (40° C./75% RH) and were examined in appropriate time points in order to control their stability.

TABLE 17
Stability data of Composition 10

| | | Stability data after 6 months | | |
|---|---|---|---|---|
| Control Tests | Limits | 25° C./60% RH | 30° C./65% RH | 40° C./75% RH |
| XRD | The XRD matches | conforms | conforms | conforms |
| | the reference | | | |
| | standard* | | | |
| Assay | 95.0-105.0% | 92.1 | 92.7 | 94.2 |
| | of the stated amount | | | |
| | of Febuxostat | | | |
| Related | Impurity I 0.2% | RRT 0.61: 0.01 | RRT 0.61: 0.02 | RRT 0.60: 0.02 |
| Substances | Impurity C 0.2% | RRT 0.74: 0.02 | RRT 0.74: 0.01 | RRT 0.74: 0.01 |
| | Impurity IX 0.2% | RRT 0.79: 0.01 | RRT 0.79: 0.01 | RRT 0.79: 0.01 |
| | Any Unknown | RRT 1.56: 0.01 | RRT 1.55: 0.01 | RRT 0.23: 0.03 |
| | NMT 0.2% (Max) | | | RRT 0.55: 0.01 |
| | | | | RRT 1.56: 0.01 |
| | Total NMT 2.0% | 0.05 | 0.05 | 0.09 |

*XRD pattern at 2-theta angles of polymorphic form C: 6.62, 10.82, 13.36, 15.52, 16.74, 17.40, 18.00, 18.70, 20.16, 20.62, 21.90, 23.50, 24.78, 25.18, 34.08, 36.72, 38.04

From all the test results demonstrated we can conclude that the preferred tablet composition of the present invention, Composition 10, exhibits the desirable dissolution rate and extent, satisfactory physical properties as well as physical and chemical stability.

While the invention has been described with reference to various specific and preferred embodiments and examples, it should be however understood that variations and modifications may be made without departing from the spirit and scope of the invention as defined in the appended claims.

1. A solid pharmaceutical composition for oral administration comprising Febuxostat and an effective amount of an alkalizing agent to improve solubility and rate of dissolution.

2. The pharmaceutical composition according to claim 1 comprising Febuxostat polymorphic form C.

3. The pharmaceutical composition according to claim 1, wherein the alkalizing agent is selected from magnesium oxide, dibasic calcium phosphate, tricalcium phosphate, calcium carbonate.

4. The pharmaceutical composition according to claim 1, wherein the alkalizing agent is magnesium oxide.

5. The pharmaceutical composition according to claim 1, wherein magnesium oxide is present in the formulation in amount 3-10%.

6. The pharmaceutical composition according to claim 1 comprising Febuxostat polymorphic form C with particle size distribution of D90<30 um.

7. The pharmaceutical composition according to claim 1, wherein it further comprises at least one pharmaceutical acceptable excipient selected from diluents, binders, disintegrants and lubricants.

8. A process for the preparation of a tablet composition comprising Febuxostat and an effective amount of an alkalizing agent to improve solubility and rate of dissolution comprising the steps of:
Sifting and mixing the internal phase excipients;
Kneading with water;
Drying the wet mass at 40° C.;
Sizing the granules;
Mixing with external phase excipients;
Adding at least one lubricant and mixing;
Compressing the resulted mixture into a tablet dosage form;
Optionally applying a film-coating on the core.

9. The process according to claim 8 comprising Febuxostat polymorphic form C with particle size distribution of \ddot{υ}90<30 μm.

10. The process according to claim 8, wherein the alkalizing agent is magnesium oxide and is comprised in amount 3-10% (w/w).
